(19) United States
(12) Patent Application Publication
(10) Pub. No.: US 2026/0041469 A1
(43) Pub. Date: Feb. 12, 2026

(54) Surgical Handpiece For Driving Orthopedic Pins And Related Accessories

(71) Applicant: Stryker Corporation, Kalamazoo, MI (US)

(72) Inventors: Kyle Boughner, Caledonia, MI (US); James Michael Chapman, Portage, MI (US); David E. Hershberger, Kalamazoo, MI (US); Erika Grace Mallery, Kalamazoo, MI (US); Caitlyn McGrail, East Grand Rapids, MI (US); Dustin James Payne, Kalamazoo, MI (US); Patrick Roach, Portage, MI (US); Theodore Uelk, Osceola, IN (US); Keith Van Liere, Holland, MI (US)

(73) Assignee: Stryker Corporation, Portage, MI (US)

(21) Appl. No.: 19/102,224

(22) PCT Filed: Aug. 9, 2023
(86) PCT No.: PCT/US2023/029904

§ 371 Date: Feb. 7, 2025

Related U.S. Application Data
(60) Provisional application No. 63/396,499, filed on Aug. 9, 2022.
(60) Provisional application No. 63/396,506, filed on Aug. 9, 2022.
(60) Provisional application No. 63/486,924, filed on Feb. 24, 2023.
(60) Provisional application No. 63/457,941, filed on Apr. 7, 2023.

(51) Int. Cl. A61B 17/88; A61B 17/00; A61B 17/16

(52) U.S. Cl. CPC A61B 17/8872; A61B 17/1628; A61B 2017/00398; A61B 2017/00424; A61B 2017/00477; A61B 2017/00734

(57) ABSTRACT

A surgical power tool assembly is provided. The assembly includes a handpiece and a pin driver attachment coupled to the handpiece. The handpiece includes a motor and a controller for operating the motor. A trigger is movably coupled to a housing of the handpiece. The controller controls a speed of the motor based on an actuated position of the trigger. An output drive is coupled to the motor and is rotated by the motor about an axis. An actuation surface of the trigger is moveable relative to the housing such that the actuation surface is closer to the axis in the actuated position than in the unactuated position. The pin driver attachment includes a housing for coupling to the handpiece. The pin driver attachment may include a lever to clamp a surgical pin to drive the surgical pin into bone.

### CROSS-REFERENCE TO RELATED APPLICATIONS

The present application claims priority to and all the benefits of U.S. Provisional Ser. No. 63/457,941, filed on Apr. 7, 2023; and Provisional Ser. No. 63/486,924, filed on Feb. 24, 2023; and Provisional Ser. No. 63/396,506, filed on Aug. 9, 2022; and Provisional Ser. No. 63/396,499, filed on Aug. 9, 2022; the entire contents of which are expressly incorporated herein by reference.

### BACKGROUND

Conventional medical and surgical procedures routinely involve the use of surgical tools and instruments which allow surgeons to approach and manipulate surgical sites. By way of non-limiting example, rotary instruments for driving surgical pins such as a surgical handpiece assembly are commonly utilized in connection with orthopedic procedures to drive surgical pins into bone. Conventional surgical handpiece assemblies have “pistol grip” configurations. In procedures where such surgical handpiece assemblies are employed, it can be difficult for a user to correctly orient the surgical handpiece to drive the surgical pin in bone without repositioning themselves or the patient.

While surgical handpiece assemblies for driving surgical pins are routinely utilized to assist in the performance of a variety of different types of medical and/or surgical procedures, there is a need in the art to continuously improve such surgical handpiece assemblies.

### SUMMARY

A first aspect of the present disclosure provides a surgical power tool assembly for driving a surgical pin such as an orthopedic pin. The surgical power tool assembly includes a surgical handpiece having: a handpiece housing, an electric motor positioned in the handpiece housing, a controller positioned in the handpiece housing, a battery removably coupled to the handpiece housing and configured to supply electrical power to the controller, a trigger moveably coupled to the handpiece housing, the trigger having an actuation surface moveable between an actuated position and an unactuated position, where the controller is configured to control a speed of the motor based on the actuated position of the trigger, and an output drive coupled to the electric motor and configured to be rotated about an axis by the electric motor, where the actuation surface is closer to the axis in the actuated position than in the unactuated position. The assembly also includes a pin driver attachment that includes: an attachment housing to be removably coupled to the handpiece housing, an actuator movably coupled to the attachment housing, a collet operable in a clamped state and an unclamped state, the collet operable to engage a surgical pin when in the clamped state, where the actuator is operatively coupled to the collet with the actuator moving between a first position and a second position, where when the actuator is in the first position, the collet is in the clamped state, and when the actuator is in the second position, the collet is in the unclamped state.

A second aspect of the present disclosure provides a surgical power tool assembly for driving a surgical pin. The surgical power tool assembly includes a surgical handpiece that having: a handpiece housing, an electric motor positioned in the handpiece housing, a controller positioned in the handpiece housing, a battery removably coupled to the handpiece housing and configured to supply electrical power to the controller, a trigger moveably coupled to the handpiece housing, the trigger having an actuation surface moveable between an actuated position and an unactuated position, where the controller is configured to control a speed of the motor based on the actuated position of the trigger, and an output drive coupled to the electric motor and configured to be rotated about an axis by the electric motor, where the actuation surface is closer to the axis in the actuated position than in the unactuated position. The assembly also includes a pin driver attachment for driving the surgical pin.

A third aspect of the present disclosure provides a surgical power tool assembly. The surgical power tool assembly includes a surgical handpiece having: a handpiece housing, an electric motor positioned in the handpiece housing, a controller positioned in the handpiece housing, a battery removably coupled to the handpiece housing and configured to supply electrical power to the controller, a trigger pivotably coupled to the handpiece housing such that the controller is configured to turn on the electric motor when the trigger is depressed and adjust a speed of the electric motor based on how far the trigger is depressed, and an output drive coupled to the electric motor and configured to be rotated about an axis by the electric motor. The assembly also includes a pin driver attachment having: an attachment housing to be removably coupled to the handpiece housing, an actuator movably coupled to the attachment housing, a collet operable in a clamped state and an unclamped state, the collet operable to engage a surgical pin when in the clamped state, where the actuator is operatively coupled to the collet with the actuator moving between a first position and a second position, where when the actuator is in the first position, the collet is in the clamped state, and when the actuator is in the second position, the collet is in the unclamped state.

A fourth aspect of the present disclosure provides a surgical power tool assembly for driving a surgical pin. The surgical power tool assembly includes a surgical handpiece having: a handpiece housing, an electric motor positioned in the handpiece housing, a controller positioned in the handpiece housing, a battery removably coupled to the handpiece housing and configured to supply electrical power to the controller, a trigger pivotably coupled to the handpiece housing such that the controller is configured to turn on the electric motor when the trigger is depressed and adjust a speed of the electric motor based on how far the trigger is depressed, and an output drive coupled to the electric motor and configured to be rotated about an axis by the electric motor. The surgical power tool assembly also includes a pin driver attachment for driving the surgical pin.

A fifth aspect of the present disclosure provides a method of inserting a surgical pin with a surgical power tool assembly. The method includes inserting the surgical pin into the coupler. The method also includes grasping the housing with a user's hand such that a user's thumb is closer to the battery than the coupler. The method also includes engaging the trigger with a user's finger while the user's thumb is closer to the battery than the coupler. The method also includes driving the surgical pin into a bone with the surgical power tool assembly.

A sixth aspect of the present disclosure provides a method of inserting a pin with a surgical power tool. The method includes grasping a housing of the surgical power tool with a user's hand. The method also includes engaging the lever with an index finger of the user's hand to clamp a surgical pin with the collet. The method also includes engaging the trigger with a finger different from the index finger of the user's hand. The method also includes driving the surgical pin into bone with the surgical power tool.

A seventh aspect of the present disclosure provides a method of driving pins in a robotically-assisted knee arthroplasty procedure. The method of driving pins also includes providing a battery-powered surgical tool. The method also includes coupling a surgical attachment to the battery-powered surgical tool. The method also includes drilling a hole in at least one bone selected from a femur, a tibia, and a patella with a drill bit using a first drive portion of the surgical attachment. The method also includes driving a first pin in one of the femur, the tibia, or the patella with a second drive portion of the surgical attachment. The method also includes driving a second pin having a different diameter than the first pin in another of the femur, the tibia, or the patella with a third drive portion of the surgical attachment.

An eighth aspect of the present disclosure provides a powered surgical instrument. The powered surgical instrument includes a housing defining an inner cavity therein, the housing including a handle portion. The instrument also includes a body portion extending distally from the handle portion. The instrument also includes a tool assembly disposed at a distal end of the body portion. The instrument also includes a control circuit and a drive motor disposed within the inner cavity, where the drive motor is mechanically coupled to the tool assembly and the control circuit is configured to control the operation of the drive motor, where the control circuit includes a plurality of field effect transistors. The instrument also includes a potting material disposed in the inner cavity encapsulating at least a portion of the field effect transistors of the control circuit.

A ninth aspect of the present disclosure provides a powered surgical instrument. The powered surgical instrument also includes a housing defining an inner cavity therein, the housing including a handle portion. The instrument also includes a body portion extending distally from the handle portion. The instrument also includes a tool assembly disposed at a distal end of the body portion. The instrument also includes a control circuit and a drive motor disposed within the inner cavity, where the drive motor is mechanically coupled to the tool assembly and the control circuit is configured to control the operation of the drive motor, where the control circuit includes a plurality of field effect transistors. The instrument also includes a metal heat sink contacting at least two of the plurality of field effect transistors.

A tenth aspect of the present disclosure provides a method for manufacturing a powered surgical device. The method includes providing an enclosure of the powered surgical device, the enclosure defining a cavity, and a control circuit disposed in the enclosure. The method also includes injecting a liquid potting material into the cavity of the sealed enclosure to hermetically seal the enclosure, the control circuit including: a circuit board; a motor controller coupled to the circuit board, and optionally, one or more field effect transistors coupled to the circuit board, and optionally one more trigger sensors and one or more motor sensors, such as one or more hall sensors. The method also includes solidifying the potting material to encapsulate at least a portion of the control circuit, such as encapsulating one more field effect transistors, the motor controller, motor sensors, trigger sensors and/or the circuit board.

An eleventh aspect of the present disclosure provides a surgical power tool. The surgical power tool includes a housing. The tool also includes an electric motor positioned in the housing. The tool also includes a coupler operatively actuated by the electric motor. The tool also includes a controller positioned in the housing. The tool also includes a battery configured to supply electrical power to the controller, the battery coupled to the housing. The tool also includes a trigger movably coupled to the housing such that the controller is configured to turn on the electric motor when the trigger is depressed and adjust a speed of the electric motor based on how far the trigger is depressed. The coupler defines an axis. The trigger defines an actuation surface and moves between an actuated position and an unactuated position. The actuation surface is closer to the axis defined by the coupler in the actuated position than in the unactuated position.

A twelfth aspect of the present disclosure provides a surgical power tool. The surgical power tool includes a housing including a barrel portion and a handle portion. The tool also includes an electric motor positioned in the housing. The tool also includes a coupler operatively actuated by the electric motor. The tool also includes a controller positioned in the housing. The tool also includes a battery configured to supply electrical power to the controller, the battery coupled to the housing. The tool also includes a trigger movably coupled to the handle portion of the housing such that the controller is configured to turn on the electric motor when the trigger is depressed. The handle portion defines a longitudinal axis. The barrel portion of the housing is operable to rotate about the longitudinal axis of the handle portion.

A thirteenth aspect of the present disclosure provides a surgical power tool. The surgical power tool includes a housing including a barrel portion and a handle portion. The tool also includes an electric motor positioned in the housing. The tool also includes a coupler operatively actuated by the electric motor. The tool also includes a controller positioned in the housing. The tool also includes a battery configured to supply electrical power to the controller, the battery coupled to the housing. The tool also includes a trigger movably coupled to the handle portion of the housing such that the controller is configured to turn on the electric motor when the trigger is depressed. The handle portion defines an axis. The barrel portion of the housing is operable to rotate about an axis of the handle portion.

A fourteenth aspect of the present disclosure provides a surgical power tool. The surgical power tool includes a housing. The housing may include a proximal region and a distal region. The housing includes a first attachment coupler in the proximal region and a second attachment coupler in the distal region. The tool also includes an electric motor positioned in the housing. The tool also includes a controller positioned in the housing. The tool also includes a battery configured to supply electrical power to the controller, the battery coupled to the housing. The tool also includes a trigger movably coupled to the housing and the controller is configured to turn on the electric motor when the trigger is depressed. The tool also includes a mode switch movably coupled to the housing and movable between a first position and a second position, and the controller is operable to control the motor in a first mode when the mode switch is in a first position and operable to control the motor in a second mode when the mode switch is in a second position. The tool also includes a first attachment configured to be driven from the motor when the first attachment is engaged with the first attachment coupler. The tool also includes a second attachment configured to be driven from the motor when the second attachment is engaged with the second attachment coupler. When the motor is in the first mode, the motor is operable to drive the first attachment and when the motor is in the second mode, the motor is operable to drive the second attachment.

A fifteenth aspect of the present disclosure provides a surgical power tool. The surgical power tool includes a housing. The tool also includes an electric motor positioned in the housing. The tool also includes a coupler operatively actuated by the electric motor. The tool also includes a controller positioned in the housing. The tool also includes a battery configured to supply electrical power to the controller, the battery coupled to the housing. The tool also includes a first trigger movably coupled to the housing such that the controller is configured to turn on the electric motor when the first trigger is depressed. The tool also includes a second trigger movably coupled to the housing such that the controller is configured to turn on the electric motor when the second trigger is depressed. The first trigger is accessible from a different grip than the second trigger.

A sixteenth aspect of the present disclosure provides a surgical power tool. The surgical power tool includes a housing. The tool also includes an electric motor positioned in the housing. The tool also includes a coupler operatively actuated by the electric motor. The tool also includes a controller positioned in the housing. The tool also includes a battery configured to supply electrical power to the controller, the battery coupled to the housing. The tool also includes a trigger movably coupled to the housing such that the controller is configured to turn on the electric motor when the trigger is depressed and adjust a speed of the electric motor based on how far the trigger is depressed. The coupler defines an axis. The trigger defines an actuation surface, the trigger moveable between an actuated position and an unactuated position. The actuation surface is closer to the axis defined by the coupler in the actuated position than the unactuated position.

A seventeenth aspect of the present disclosure provides a powered surgical tool. The powered surgical tool includes a handpiece defining a void space. The tool also includes an electric motor disposed in the void space. The tool also includes a first printed circuit board disposed in the void space and being rigid, the first circuit board including trigger sensors and motor control sensors. The tool also includes a second printed circuit board disposed in the void space and being rigid. The tool also includes a third circuit board disposed in the void space and being rigid. The tool also includes each of the first circuit board, the second circuit board, and third circuit board defining a plane, with the planes of the first, second and third circuit boards being parallel to one another. The tool also includes a controller configured to regulate power drawn from a power source based on user input, the controller being mounted to one of the second printed circuit board and the third circuit board.

An eighteenth aspect of the present disclosure provides a surgical power tool assembly for driving a surgical pin. The surgical power tool assembly includes a surgical handpiece having a handpiece housing. The assembly also includes an electric motor positioned in the handpiece housing. The assembly also includes a controller positioned in the handpiece housing. The assembly also includes a trigger moveably coupled to the handpiece housing, the trigger having an actuation surface moveable between an actuated position and an unactuated position, where the controller is configured to control a speed of the motor based on the actuated position of the trigger. The assembly also includes an output drive coupled to the electric motor and configured to be rotated about an axis by the electric motor. The actuation surface is closer to the axis in the actuated position than in the unactuated position. The assembly also includes a pin driver attachment for driving the surgical pin.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a surgical system including a surgical handpiece and a surgical attachment.

FIG. 2 is another perspective view of the surgical system including the surgical handpiece and the surgical attachment.

FIG. 3 is an elevation view of the surgical system.

FIG. 4 is a section view of the surgical system with a lever of the surgical attachment shown in a first position.

FIG. 5 is a section view of the surgical system with the lever of the surgical attachment shown in a second position.

FIG. 6 is a section view of the surgical handpiece of the surgical system with a trigger in a first position.

FIG. 7 is a section view of the surgical handpiece of the surgical system with the trigger in a second position.

FIG. 8 is an exploded view of the surgical handpiece.

FIG. 9 is a perspective view of a surgical attachment of the surgical system and a pin.

FIG. 10 is a section view of the surgical attachment from FIG. 9 and the pin.

FIG. 11 is a section view of the surgical attachment from FIG. 9.

FIG. 12 is a section view of an alternative implementation of the surgical attachment of the surgical system.

FIG. 13 is an elevation view of a pin driver attachment of the surgical system.

FIG. 14 is a sectional view of a first implementation of the pin driver attachment.

FIG. 15 is a section view of a second implementation of the pin driver attachment.

FIG. 16 is a section view of a third implementation of the pin driver attachment.

FIG. 17A is a perspective view of a printed circuit board assembly.

FIG. 17B is another perspective view of the printed circuit board assembly.

FIG. 18 is another perspective view of the printed circuit board assembly.

FIG. 19 is an elevation view of the printed circuit board assembly in another configuration.

FIG. 20 is a perspective view of the printed circuit board assembly including a heat sink.

FIG. 21 is a perspective view of the printed circuit board assembly including another heat sink.

FIG. 22 is a perspective view of another configuration of the printed circuit board assembly including a potting material.

FIG. 23 is a side elevation view of one configuration of the printed circuit assembly disposed in an enclosure of a surgical handpiece.

FIG. 24 is a perspective view of another implementation of a surgical power tool assembly having multiple surgical attachments.

FIG. 25 is a perspective view of another implementation of a surgical power tool assembly having multiple surgical attachments that may be configured in different orientations.

FIG. 26 is a perspective view of another implementation of a surgical power tool assembly having multiple surgical attachments that may be configured in different orientations.

FIG. 27A is a perspective view of another implementation of a surgical power tool assembly in a first configuration.

FIG. 27B is a perspective view of the implementation of the surgical power tool assembly of FIG. 27A in a second configuration.

FIG. 27C is a perspective view of the implementation of the surgical power tool assembly of FIGS. 27A and 27B in a third configuration.

FIG. 28 is a perspective view of another surgical power tool assembly.

FIG. 29A is an elevation view of another surgical power tool assembly.

FIG. 29B is an elevation view of another surgical power tool assembly.

FIG. 30A is a side elevation view of another configuration of the surgical power tool assembly.

FIG. 30B is a side elevation view of another configuration of the surgical power tool assembly.

FIG. 31 is a perspective view of another configuration of surgical power tool assembly.

FIG. 32 is a sectional view of the surgical power tool assembly of FIG. 31.

FIG. 33 is a perspective view of another configuration of the surgical power tool assembly.

FIG. 34 is a sectional view of the surgical power tool assembly of FIG. 33.

### DETAILED DESCRIPTION

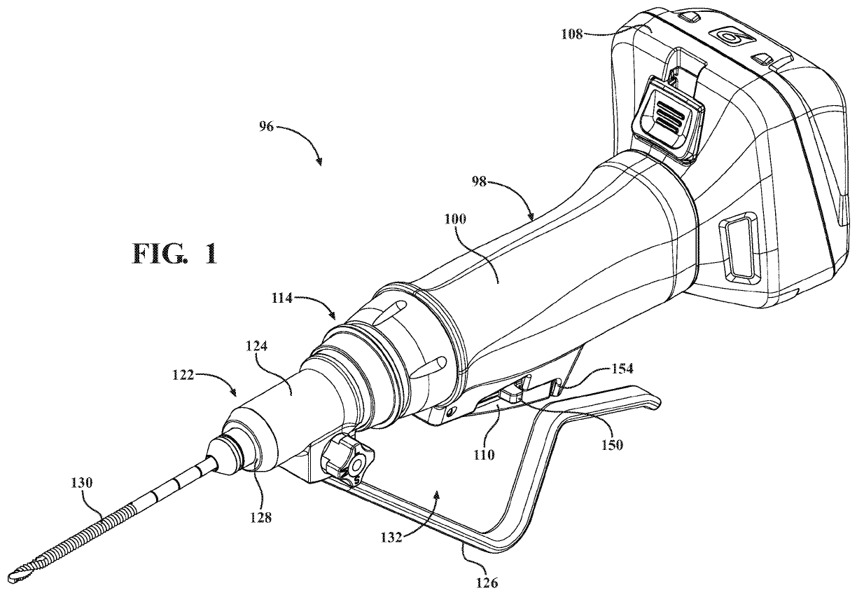

With reference to FIG. 1, a surgical power tool assembly 98 of a surgical system 96 is contemplated. The surgical power tool assembly 98 may be referred to as a surgical handpiece assembly. An exemplary surgical power tool assembly is described in U.S. Patent Publication No. 2016/0206327. The power tool assembly may include a surgical handpiece having a housing 100, an electric motor 102 positioned in the housing 100, and an output drive 104 operatively coupled to and actuated by the electric motor 102 such that the output drive 104 is rotated by the motor 102 about an output drive axis AX. The output drive 104 may be configured to engage any suitable surgical end effector such as a surgical pin or wire, or a drill bit, or a driver. The power tool assembly may further include a controller 106. The controller 106 may be optionally positioned within the housing 100. The surgical power tool assembly may optionally include a removable battery 108 that is configured to supply electrical power to the controller and to the electric motor 102. One battery that can be employed with this version of the invention is described in U.S. Patent App. Pub. No. 2007/0090788 published on Apr. 26, 2007, and herein incorporated by reference. The battery 108 may be configured to be coupled to the housing via a twist lock design. One suitable example of such a battery is described in U.S. Pat. No. 11,534,181, issued on Dec. 27, 2022, and herein incorporated by reference in its entirety. A trigger 110 may be movably coupled to the housing 100, and suitable trigger sensors may be included in the tool such that the controller 106 is configured to turn on the electric motor 102 and/or adjust the speed of the motor 102 and in turn the output drive 104 based on the extent that the trigger 110 is actuated and/or depressed. The output drive 104 may include a gearset 112 disposed between the motor 102 and a tool coupler 114 of the output drive 104 to increase torque available at the tool coupler 114. In some configurations, the tool coupler 114 may be used to secure surgical pins and/or drill bits to the handpiece, in other configurations, the tool coupler 114 may be used to secure one or more of the surgical attachments to the handpiece. In one configuration, the controller 106 is configured to regulate power drawn from a power source (e.g., the battery) based on user input on the trigger 110. In some configurations, the trigger 110 is pivotably coupled to the housing 100.

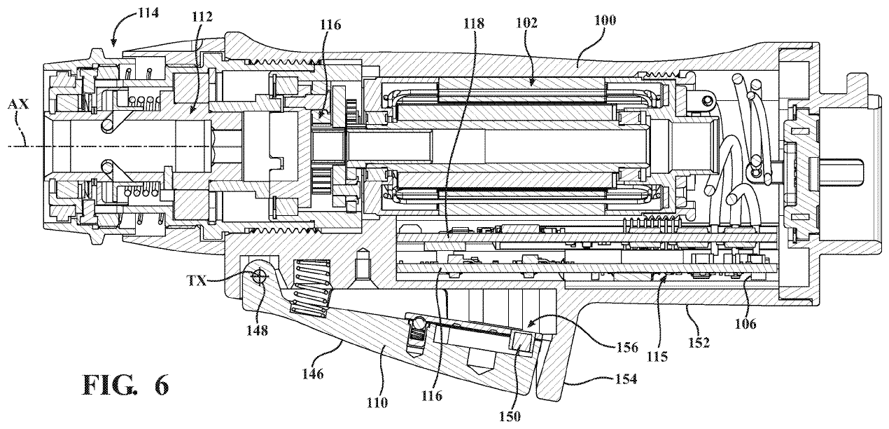

The handpiece housing 100 may define a void space for receiving the motor 102. A printed circuit board assembly 115 may be disposed in the void space. The printed circuit board assembly 115 may include first, second, and third printed circuit boards 116, 118, 120. The first circuit board 116 may include the trigger sensors and motor control sensors. Each of the first, second, and third printed circuit boards 116, 118, 120 may be rigid. Further, each of the first circuit board, the second circuit board, and the third circuit board 116, 118, 120 may define a plane. The planes of each of the first, second, and third circuit boards 116, 118, 120 may be parallel to one another and disposed on separate planes such that the printed circuit boards 116, 118, 120 may not be considered to be coplanar. The controller 106 may be mounted to one of the second printed circuit board and the third circuit board 118, 120. The first, second, and third printed circuit boards 116, 118, 120 may be connected to one another via one or more flex connectors. In other configurations (see FIGS. 6-8), the printed circuit board assembly 115 may comprise only first and second printed circuit boards 116, 118.

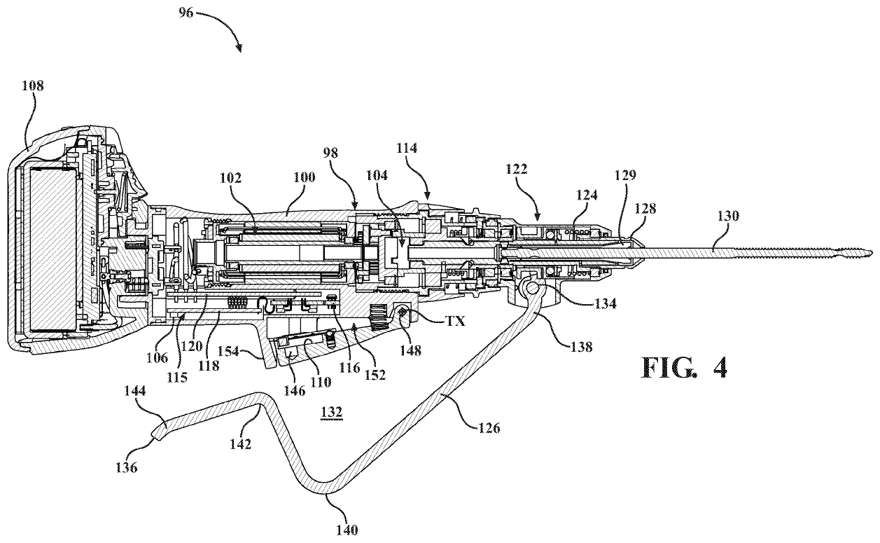

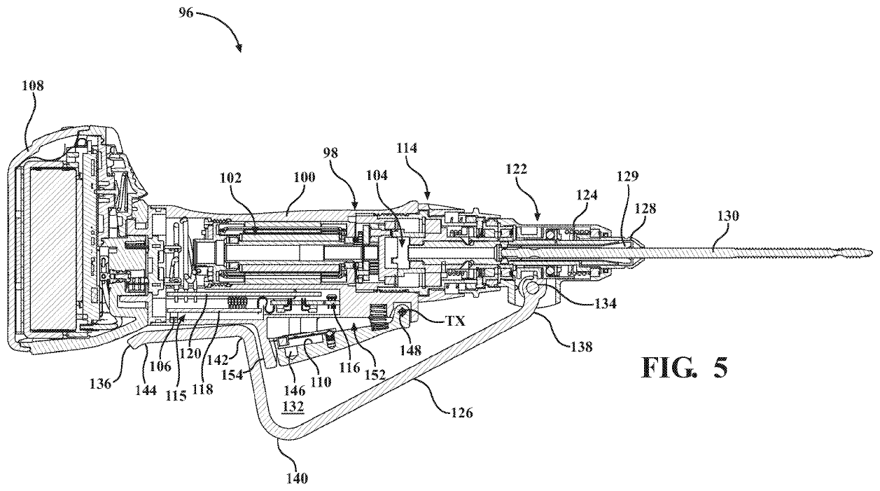

A range of surgical attachments may be used with the surgical power tool assembly 98 contemplated herein. One such surgical attachment is a pin driver attachment 122. The pin driver attachment 122 includes an attachment housing 124 to be removably coupled to the housing 100 of the power tool. The pin driver attachment 122 may also include an actuator 126 movably coupled to the attachment housing 124. The actuator 126 may be realized as a lever or a twist collar. The pin driver attachment 122 may further include a collet 128 attached to the attachment housing. The collet 128 is operable in a clamped state (FIG. 5) and an unclamped state (FIG. 4). The collet is operable to engage a surgical pin 130 when in the clamped state and to permit the pin 130 to move relative to the collet 128 in the unclamped state. The actuator 126 is operatively coupled to the collet 128. The actuator 126 is moveable between a first position and a second position. In the first position, the collet 128 is in the clamped state. In the second position, the collet 128 is in the unclamped state. Suitable examples of a pin driver attachment 122 with similar collet designs for gripping pins in response to movement of an actuator are described in International Patent Application No. PCT/US2019/054093, which is hereby incorporated by reference in its entirety.

As shown in FIGS. 4 and 5, the collet 128 may include one or more jaws 129 movably coupled to the attachment housing. The one or more jaws may be moveable relative the attachment housing 124 in response to the position of the actuator 126 (e.g., the lever) such that the one or more jaws 129 in the clamped state of the collet 128 are closer to the output drive axis AX to engage the surgical pin than the one or more jaws 129 in the unclamped state of the collet.

As noted above, the actuator 126 may be realized as a lever 126. The lever 126 may define a recess 132. The trigger 110 may be aligned with the lever 126 and the recess 132 such that actuation of the trigger 110 within the recess 132 and actuation of the lever 126 occur in a single reference plane. The lever 126 may define a pivot end 134 and free end 136. The lever 126 may also comprise a first bend 138, a second bend 140, and a third bend 142. The first bend 138 and the second bend 140 may be disposed closer to the pivot end 134 than the free end 136. The first bend 138 and second bend 140 may be acute. The first bend 138 facilitates establishment of the recess 132 for placement of some of the user's hands. The second bend 140 facilitates bringing the free end 136 of the lever 126 closer to the housing 100 so that the user can easily grasp and actuate the lever 126. The third bend 142 may be obtuse. The third bend 142 provides an easy purchase for one or more of the user's fingers while holding the housing 100 in their palm. The lever 126 may further comprise a fourth bend 144. The fourth bend 144 may be closer to the free end 136 than the third bend 142. The fourth bend 144 may be obtuse. The fourth bend 144 may curve away from the housing 100 so that the user's fingers do not slip off the lever 126 between the third and fourth bends 142, 144. Collectively, the bends 138, 140, 142, 144 in the lever 126 help facilitate placement of a portion of the user's hand in the recess 132 of the lever 126 so that the user may simultaneously operate the trigger 110 and the lever 126 with a neutral hand placement. The neutral hand placement is more ergonomic than conventional pistol grip designs.

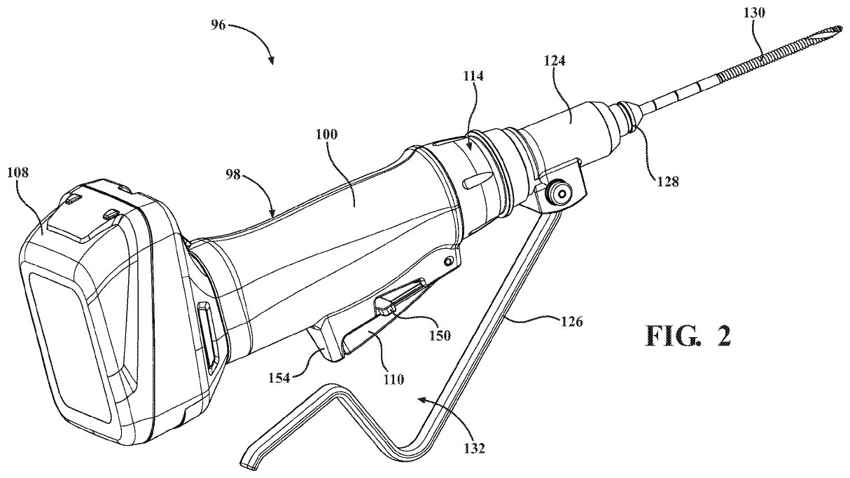

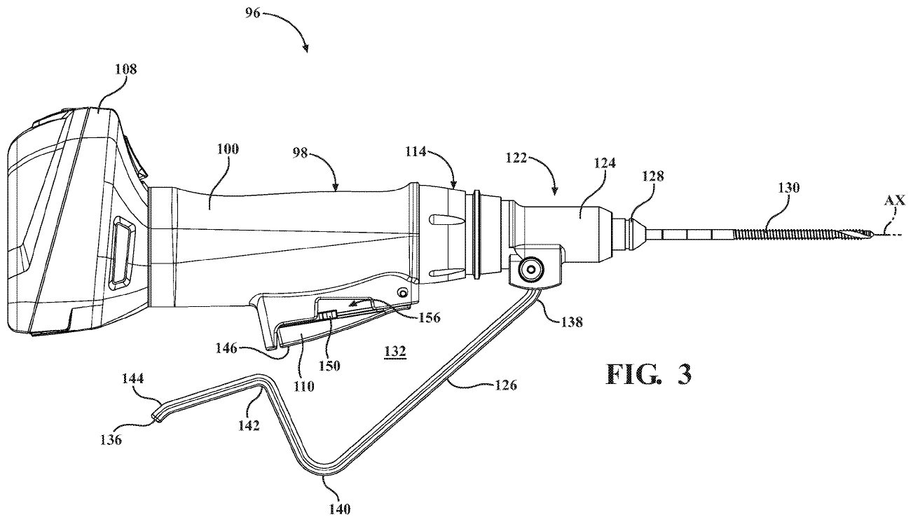

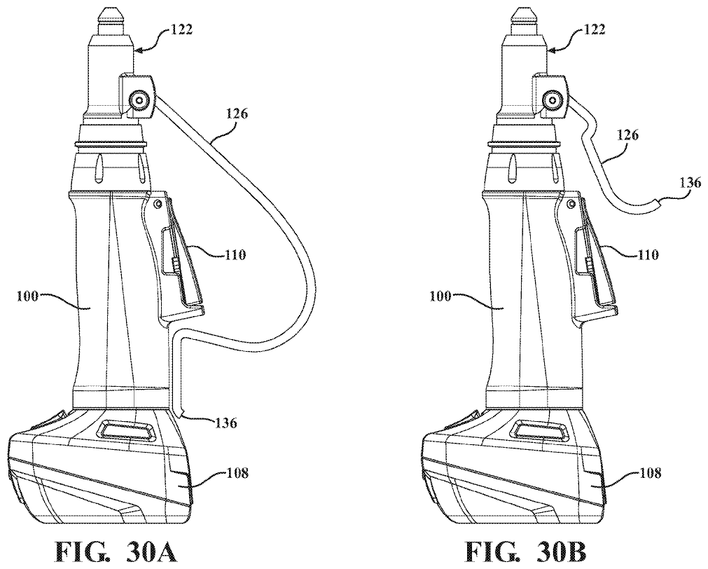

Referring to FIGS. 30A and 30B, alternative configurations of the lever 126 are shown. The lever 126 in FIG. 30A is comparable to the configuration of the lever 126 shown in FIGS. 1-5. Specifically, the free end 136 of the lever 126 is disposed proximal the trigger 110. The lever 126 in FIG. 30B is shorter by comparison. At least a portion of the trigger 110 is disposed proximal the free end of the lever 126 in FIG. 30B.

A method of inserting a pin with a surgical power tool assembly 98 is also disclosed. The method comprises depressing the lever 126 with an index finger and depressing the trigger 110 with a finger different from the index finger. Operation of the electric motor 102 and in turn the output drive 104 of the power tool 98 may be controlled based on the position of the trigger 110. The collet 128 may be operable between the clamped and unclamped states based on the position of the lever 126. The user grasps the housing 100 of the surgical power tool assembly 98 with a hammer grip. Said differently, the user may grasp the housing 100 such that user's thumb is closer to proximal end of the tool 98 and/or the battery 108 than a distal end of the tool 98.

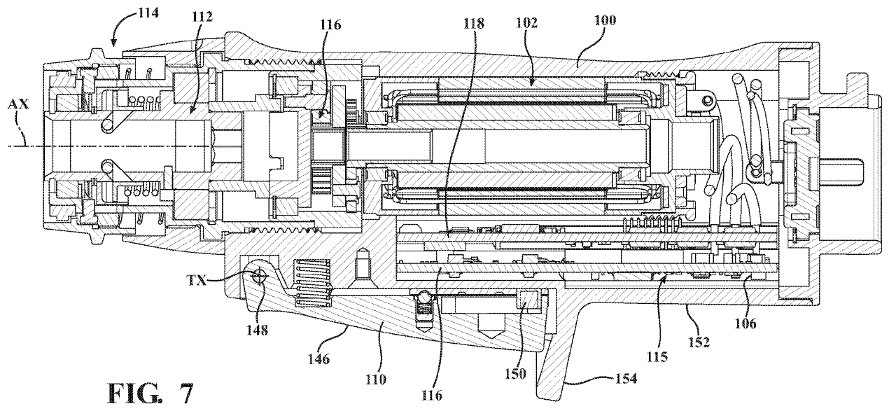

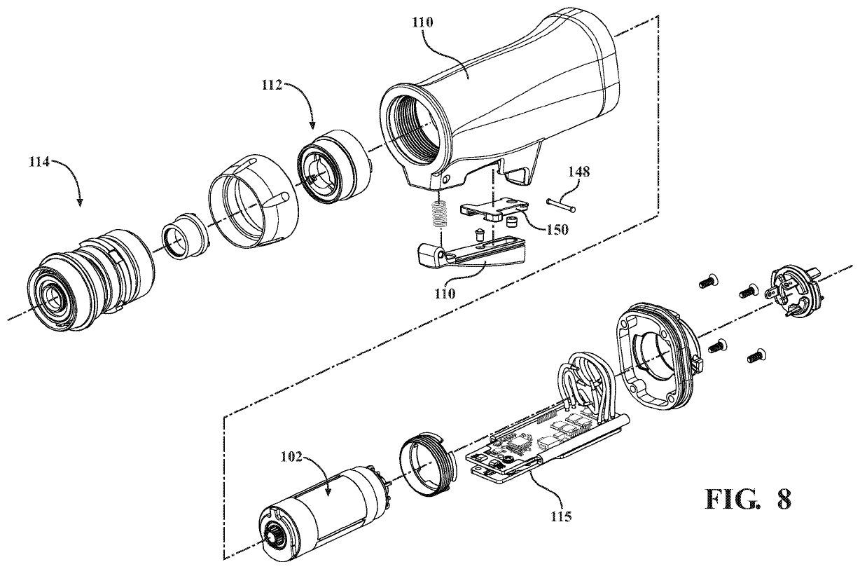

The trigger 110 may define an actuation surface 146 to be engaged by one or more digits of a user's hand. The actuation surface 146 moves between an actuated position (FIG. 7) and an unactuated position (FIG. 8) during use of the trigger 110. In the actuated position, the controller 106 is configured to control a speed of the motor 102 or otherwise energize the motor 102 to rotate the output drive 104. In the unactuated position, the controller 102 may be configured to turn the motor 102 off so that the output drive 104 is not driven to rotate by the motor 102. The surgical power tool assembly 98 is constructed such that the actuation surface 146 is closer to a coupler and/or output drive axis AX in the actuated position than in the unactuated position. This inline trigger configuration can provide for more convenient hand positioning while the surgical tool 98 is used for different surgical applications, such as in placing surgical pins. The trigger 110 may be pivotable relative to a trigger axis TX. The trigger axis TX may be optionally defined by a trigger mounting pin 148. The trigger 110 may comprise a pivoting end connected to the mounting pin and a free end that is pivotable about the trigger axis TX.

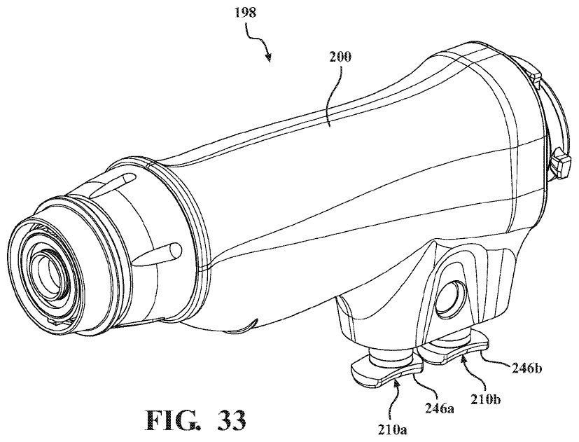

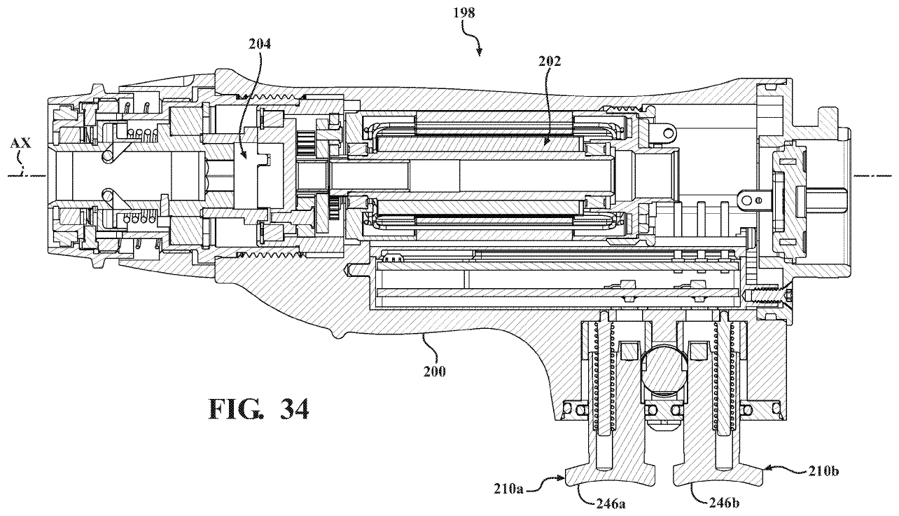

Another configuration of the surgical handpiece assembly 198 is shown in FIGS. 33 and 34. In this configuration, the surgical handpiece assembly 198 includes a first trigger 210a and a second trigger 210b. The first trigger 210a has a first actuation surface 246a. The first trigger 210 is configured to cause the electric motor to rotate the output drive 204 about the axis AX in a first direction. The second trigger 210b has a second actuation surface 246b. The second trigger 210b may be configured to cause the electric motor 202 to rotate the output drive 204 about the axis AX in a second direction opposite the first direction. In the configuration illustrated in FIGS. 33 and 34, the first and second triggers 210a, 210b are slidably coupled to the housing 200. In other configurations, the first and second triggers 210a, 210b may be pivotably coupled to the housing 200.

Returning to FIGS. 1-8, the trigger 110 may include a run-safe switch 150 that is operably connected to the controller 106. The run-safe switch 150 may be displaceable between at least a first position and a second position. In the first position, operation of the trigger 110 to pivot about the trigger axis TX may not cause actuation of the motor 102 and in turn the output drive 104 to rotate the pin. In the second position, operation of the trigger 110 to pivot about the trigger axis TX may cause actuation of the motor 102 and in turn the output drive 104 to rotate the pin. In other configurations, the run-safe switch 150 may be moveable to a third position to rotate the pin in an opposite direction.

The housing 100 may include a base 152 and a protrusion 154 extending from the base 152. The protrusion 154 may be disposed adjacent the trigger 110 and may extend farther away from the coupler axis AX than the actuation surface 146 is from the coupler axis AX when the trigger 110 is in the actuated position or the unactuated position. The protrusion 154 may prevent accidental actuation of the trigger 110 when the surgical power tool assembly 98 is resting on a working surface, e.g., if the user sets the surgical tool 98 down and the tool rotates slightly, the protrusion would 154 prevent movement of trigger 110 under the weight of the surgical tool 98 or external forces. The protrusion 154 may define a cut out 156 for receiving the run safe switch 150 in one or more positions, such that the run safe switch 150 does not impede movement of the trigger 110 relative to the housing 100.

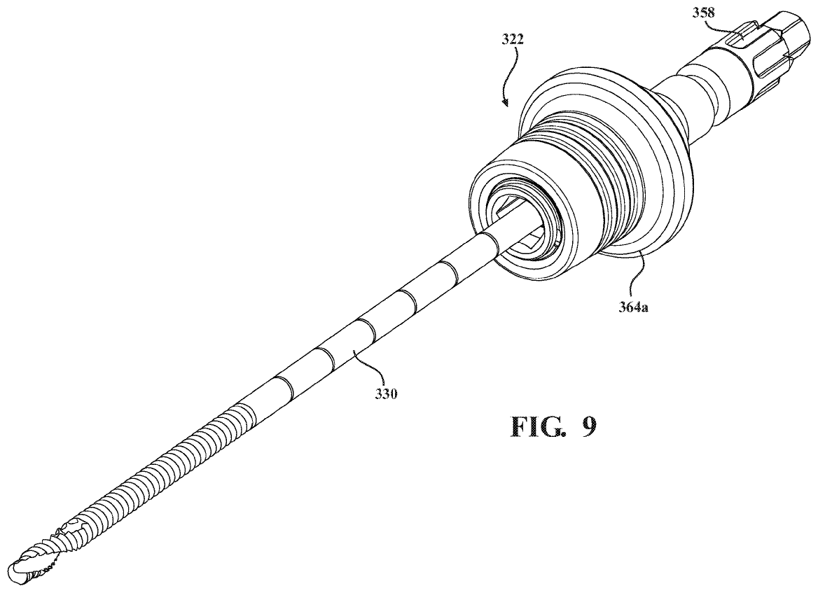

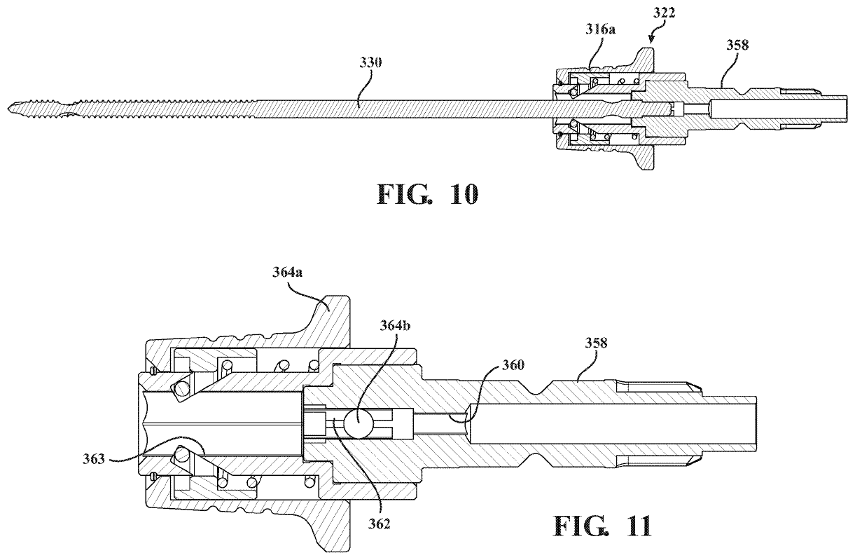

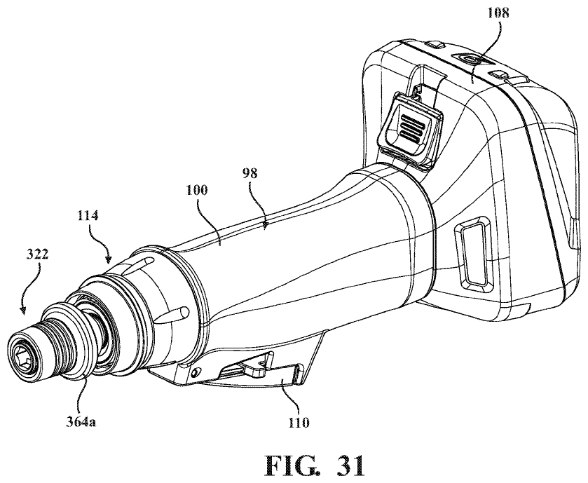

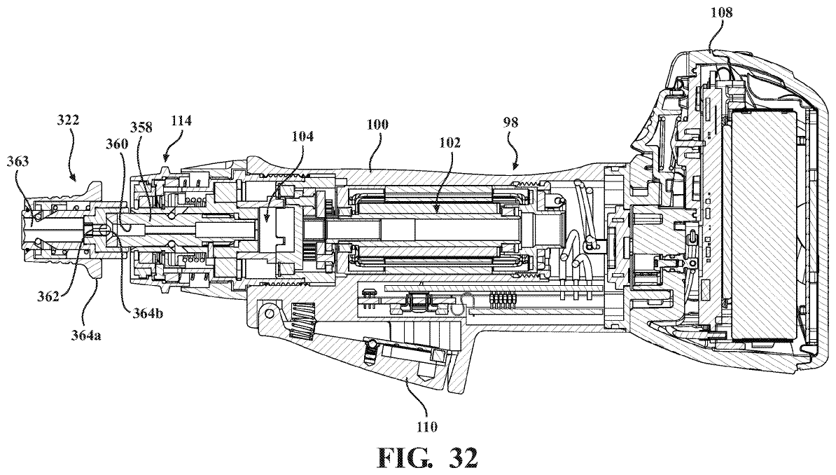

Referring to FIGS. 9-11, another configuration of the pin driver attachment 322 is illustrated. The pin driver attachment may include an input shaft 358 that defines features for receiving torque from the output drive 304 of the handpiece 298. The pin driver attachment may include a first drive portion having a first bore 360, a second drive portion having a second bore 362, and a third drive portion having a third bore 363. The first bore 360 may be coaxial with the second bore 362 and the third bore 363. The first bore 360 may be smaller than (e.g., have a different or smaller cross-sectional area) the second bore 362 and the second bore 362 may be smaller than the third bore 363. One or more tool couplers 364a, 364b may be coupled to the input shaft 358. The tool couplers 364a, 364b are movable between an engaged position and an unengaged position. In the configuration illustrated in FIG. 11, one of the tool couplers 364a comprises a collar to secure a pin to the third bore 363 and another tool coupler 364b comprises one or more magnets, offset to the center of the first and second bores 360, 362 to secure a pin including ferrous material to the pin driver attachment 322 in the first and second bores 360, 362. In the engaged position, a drill bit or pin can be secured in the tool couplers 364a, 364b. The third driver portion may be used to drive a twist drill. The first and second drive portions may be used to drive the two different surgical pins. In the unengaged position, the drill bit or pin can be released from the tool couplers 364a, 364b. The first bore 360 may include a plurality of flats for driving the drill bit or the surgical pin. The second bore 362 may include a plurality of flats for driving the drill bit or the surgical pin. Referring to FIGS. 31 and 32, the pin drive attachment 322 is shown being coupled to the surgical power tool assembly 98 instead of the pin drive attachment 122 shown in FIG. 1.

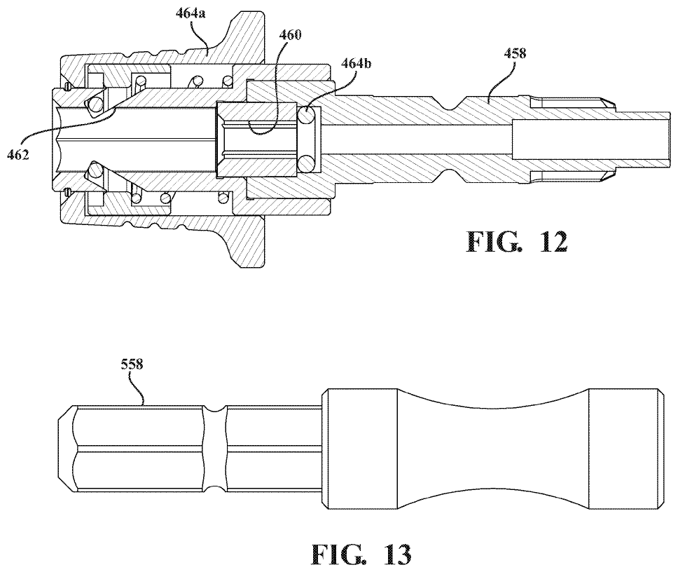

Referring to FIG. 12, another pin driver attachment 422 is provided. The pin driver attachment 422 may include an input shaft 458 that defines features for receiving torque from the output drive 404 of the handpiece 398. The pin driver attachment may also include a first bore 460 and a second bore 462. The first bore 460 is coaxial with the second bore 462. The first bore 460 is smaller than the second bore 462. One or more tool couplers 464a, 464b are secured to the input shaft 458. The tool couplers 464a, 464b are movable between an engaged position and an unengaged position. One of the tool couplers 464a comprises a collar to secure a pin to the second bore 462 and another tool coupler 464b comprises an O ring to secure the pin disposed in the first bore 460. In the engaged position, a drill bit or a pin can be secured in the tool couplers 464a, 464b. In the unengaged position, the drill bit can be released from the tool coupler 464a, 464b. The first bore 460 may include a plurality of flats. The second bore may include a plurality of flats. A surgical pin that includes at least six flats may be received by the second bore.

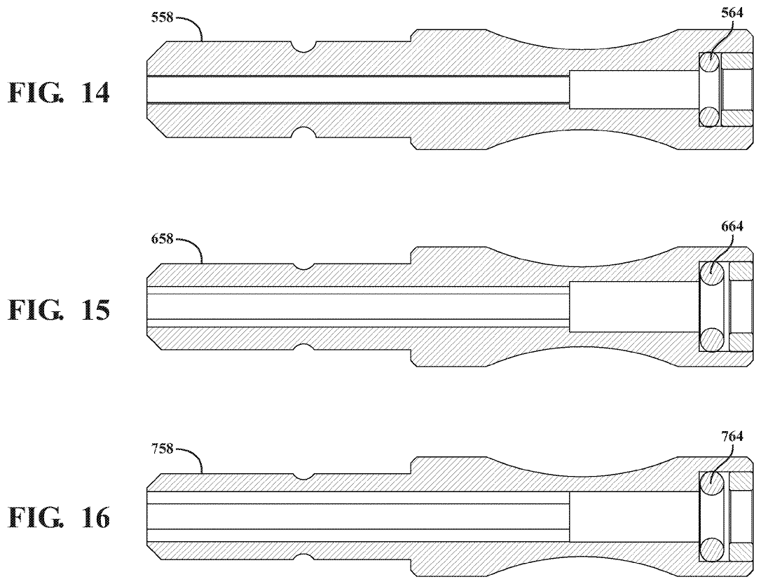

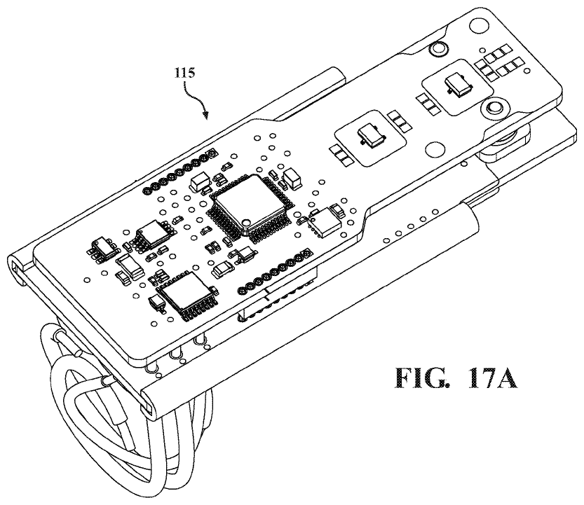

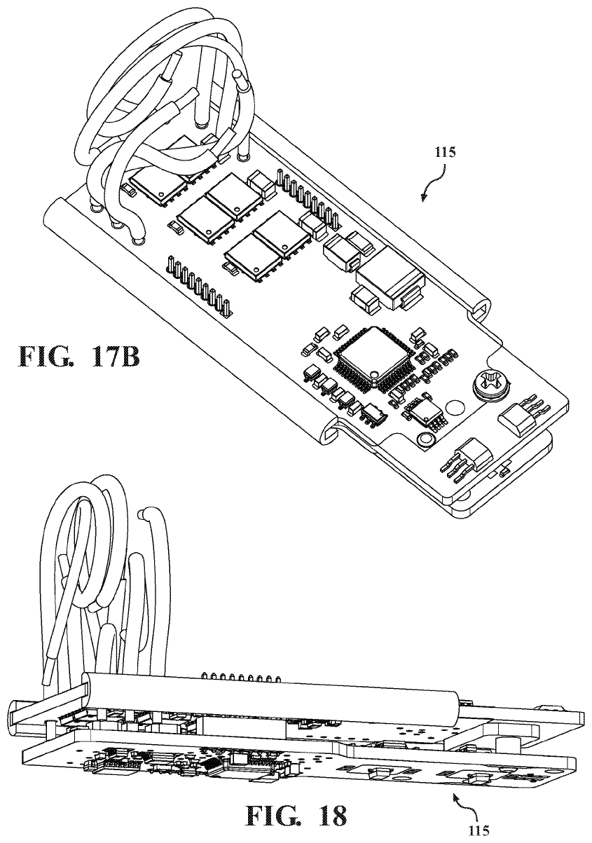

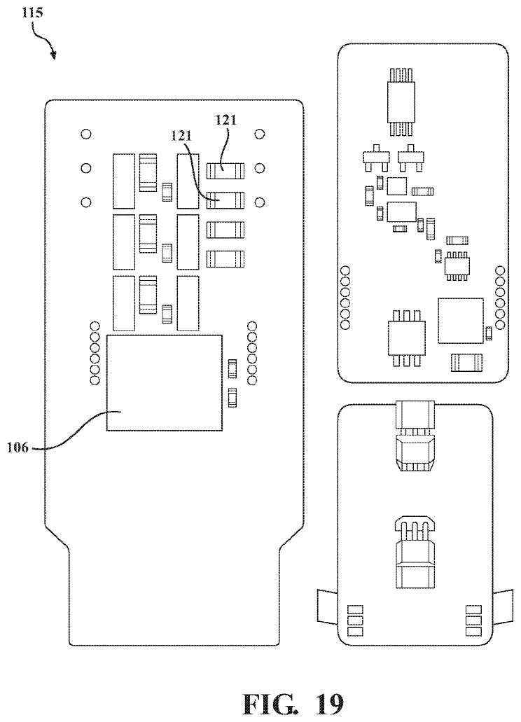

FIGS. 13-16 show a variety of pin driver attachments having identical input shafts 558, 658, 758 and different bores to receive pins having different diameters and/or coupling configurations. Using these attachments promotes a better workflow by providing uniform coupling of identical input shafts 558, 658, 758 of the different pin driver attachments while allowing for attachment of different pins to the different pin driver attachments shown in FIGS. 13-16. The input shafts 558, 658, 758 may be coupled to the surgical attachments 322, 422 or to the tool coupler 114 described above.

A method of driving pins in a robotically-assisted knee arthroplasty procedure is also contemplated. The method includes providing the battery to the surgical power tool assembly 98. The surgical attachment 122, such as the pin driver attachment 122, is coupled to the surgical power tool assembly 98. A hole is drilled in a femur, a tibia, or a patella of a patient with a first drill bit using the surgical attachment 122. A first pin is driven in the femur, the tibia, or the patella with the surgical attachment. A second pin is driven in one of the other of the femur, the tibia, and the patella with the surgical attachment. The first pin may have a different diameter than the second pin. The pins may be used to secure navigation trackers (not shown) to bones of the patient. Using the same surgical attachment 122 for each of the pins helps to make workflow more efficient by saving time during a procedure by not having to use multiple surgical attachments and reducing the number of components that require sterilization. Furthermore, the stack-up of components is smaller, which decreases the distance between where the pin/tool/drill bit is coupled to the surgical power tool assembly 98 and where the user grasps the surgical power tool assembly 98.

Another method of inserting a pin is also contemplated. The method includes grasping the housing 100 of the surgical power tool assembly 98 with a user's hand. The user may grasp the housing 100 with a hammer grip. The user may grasp the housing 100 such that the user's thumb is closer to a proximal end of the surgical power tool assembly 98 than a distal end of the surgical power tool assembly 98. The method further includes engaging the lever 126 with an index finger of the user's hand to clamp a surgical pin with the collet 128. The method further includes engaging (e.g., by depressing) the trigger 110 with a finger different from the index finger of the user's hand. The method also includes driving the surgical pin into bone with the surgical power tool assembly 98. The user may engage the lever 126 with the index finger while the user's thumb is closer to a battery 108 of the surgical power tool assembly 98 than the index fingers are from the battery 108.

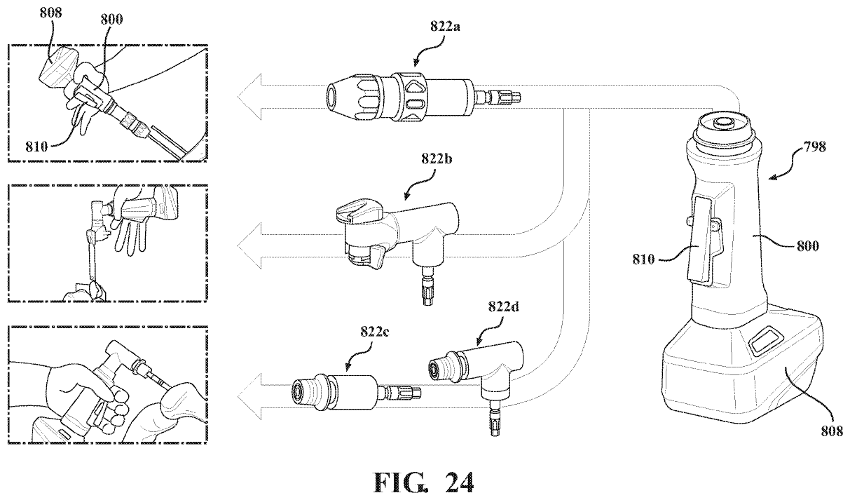

Another method of inserting a pin is also contemplated. The method may include inserting the surgical pin into a coupler of the surgical power tool assembly 98. The method further includes grasping the housing 100 of the power tool 98 such that the user's thumb is closer to the battery 108 than the tool coupler 114. The method may further include engaging (e.g., depressing) the trigger 110 with the user's hand while the user's thumb is closer to the battery 108 than the tool coupler 114. The method further includes driving the surgical pin into the bone with the surgical power tool assembly. The method may further include depressing the lever 126 with the user's finger while the user's thumb is closer to the battery 108 than the coupler. The steps of depressing the lever 126 and depressing the trigger 110 may be performed with different fingers. 45. The step of grasping the housing may further include grasping the housing 100 such that the battery 108 is disposed above the user's hand during use (see FIG. 24).

While the methods described above are with reference to the surgical power tool assembly 98 described above, it is appreciated that the methods could also apply to the other surgical power tool assemblies described herein.

The motor 102 may be a brushless DC motor. The surgical tool 98 may include a 3-phase H-bridge to commutate the brushless DC motor. The H-Bridge may control the speed and direction of the powered motor.

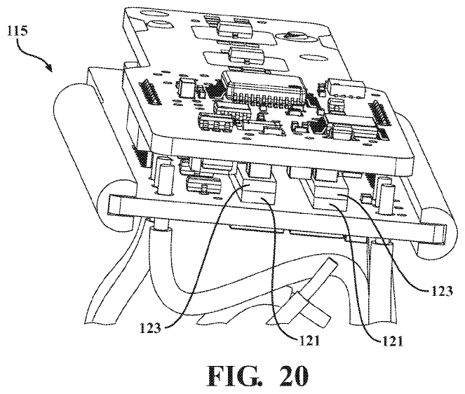

Referring to FIG. 20, one or more of the printed circuit boards 116, 118, 120 may comprise field-effect transistors 121 (FETs). The FETs 121 may be driven by a generic gate driver. The gate driver may have a maximum gate sink and source current of 2 Amps. This gate driver supplies a gate-source voltage of 5 Volts so as not to exceed a gate-source rating of the FETs 121. The gate driver may also have internal bootstrap capacitors for driving the high-side FETs 121. The FETs 121 may comprise Gallium-Nitride FETs. These FETs 121 have a characteristically low input capacitance that allows for high switching speeds. The one or more printed circuit boards may be manufactured from a fiberglass material, such as FR-4, or other suitable construction.

For certain motor applications, the end-user desires a surgical tool 98 where the space available for the motor control electronics can be heavily restricted. This could be due to the position of the motor or other application specific restraints. However, when the surgical power tool assembly 98 is used under a heavy power load, a significant amount of heat can be generated due to the high current needed to sustain a high-torque application.

Various components of the printed circuit boards 116, 118, 120 may be soldered. Exemplary solder may be high temperature solders having a melting point lower than 280, 290, or 300 degrees Celsius. In some instances, one or more of the printed circuit boards 116, 118, 120 may include one or more metal heat sinks 123 to absorb heat produced from the heavy power load. The metal heat sinks 123 may be selected from various metals and metal alloys. For example, the metal heat sinks may comprise one or more materials selected from a group consisting of Aluminum 1050, Aluminum 1070, Aluminum 1100, Aluminum 1200, or Aluminum 1370. Exemplary aluminum alloys include alloys including copper, manganese, zirconium, and/or titanium. The heatsink material may be one having a rating of at least 200 W/mK, or at least 220 W/mK, or at least 230 W/mK.

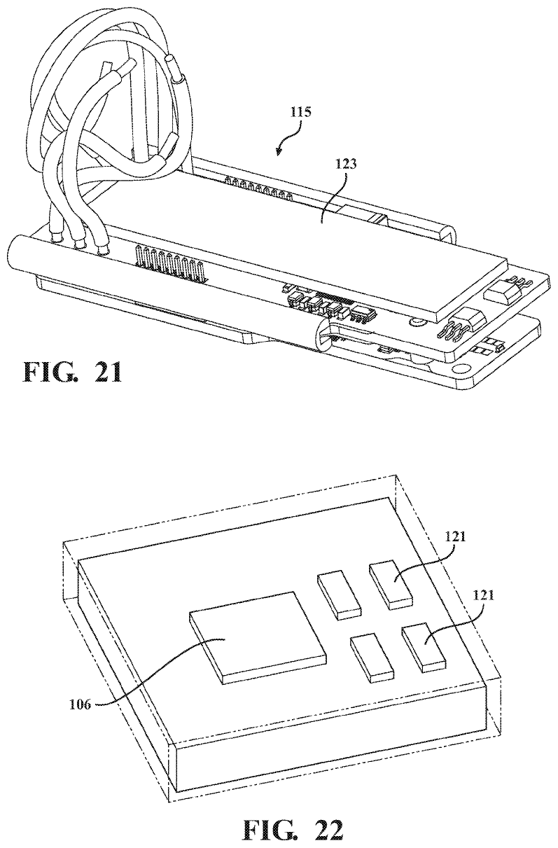

Referring to FIGS. 20 and 21, the metal heat sink 123 may be located below the board including the FETs 121, above the board including the FETs 121, or the metal heat sink 123 may be located on top and bottom of the board including the FETs 121.

The printed circuit board assembly 115 may include a dual-heatsink solution that includes an aluminum heat sink 123 that runs across and in direct contact with the high and low side transistors respectively. In one specific implementation, the dual-heatsink solution comprises two Aluminum 1370 heatsinks that are 16.05 mm×2.3 mm×1 mm in direct contact with the GaN FETs. The surgical power tool assembly 98 may include a metal heat sink 123 for each of the three FETs 121. In other implementations, the heat sinks 123 may comprise other dimensions and still be in direct contact with the FETs 121.

One or more of the metal heatsinks 123 may be used in combination with a thermally conductive potting material, such as a silicone elastomer potting material. The potting material may be positioned between two of the printed circuit boards, such that the potting material directly contacts the printed circuit board that includes the FETs. Exemplary potting materials may comprise one or more materials selected from a group consisting of Sylgard 184, Sylgard3-6605, Sylgard 160, DowSil TC-6020, or DowSilTC4025. In some implementations, suitable potting materials may have a thermal conductivity of at least 0.25, at least 0.5, at least 0.75, least 1, or at least 2.5 W/mK.

The potting material may include a thermally conductive additive. The thermally conductive additive may be selected from a group consisting of abrasive ceramics, lubricious ceramics, boron nitride, aluminum oxide, aluminum nitride, and combinations thereof.

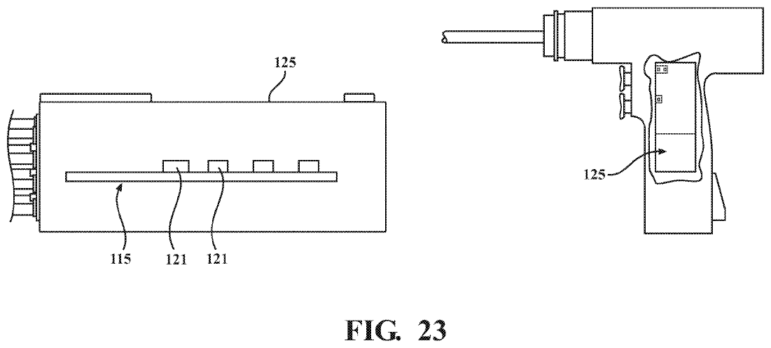

Referring to one exemplary implementation illustrated in FIG. 23, The housing of the surgical handpiece 98 may define an inner cavity, such as a separate enclosure 125, in which the control circuit (such as one or more of the printed circuit boards) and/or the motor as well as other components of the instrument are disposed. The potting material may be injected into the cavity so that the material flows into and through the cavity, thereby coating and encapsulating one or more internal components (e.g., the control circuit or components thereof) of the instrument. The potting material may be injected such that the cavity is either partially or wholly filled with the material. Encapsulation of the internal components may mitigate void space within the inner cavity. Further, the material may seal the components, thereby providing protection from moisture, chemical compounds (e.g., cleaners), vapors, gases, and biological contaminants. This manner of sealing also allows for sterilization of the instrument to provide the option for multiple uses of the instrument.

Prior to injecting the potting material into the housing or enclosure, the housing or enclosure may be suitably sealed to withstand pressures of the potting injection process as well as to hermetically seal the housing. The potting material may be any material that may be any liquid or amorphous material that solidifies upon injection into the cavity. Once solidified, the material may be relatively rigid to protect the components from shock, vibration, maintain compliance, and to reduce stress under extreme temperatures and other environmental conditions. The potting material may also take the form of a solid in some configurations.

Referring to FIG. 23, the one or more printed circuit boards may be provided in a control enclosure 125. The enclosure may be a control module for regulating operation of a motor integral with the powered surgical tool. The enclosure may define a shell formed with an opening, and the control circuit may be disposed within the shell. One or more conductive pins may extend through corresponding openings of the enclosure.

The one or more printed circuit boards may include one or more sensors that monitor the states of externally mounted triggers. Attached to each trigger and located inside the tool housing is a magnet. Internal to the control module are optionally magnetic field sensors. Each sensor generates a varying signal as a function of the proximity of an associated one of the trigger magnets. The manual displacement of the trigger results in a like displacement, inside the tool, of the magnet. When a trigger and magnet are so displaced, the complementary sensor generates a signal that indicates the movement has occurred. Upon receipt of this signal, the control circuit generates the signal needed to allow an energization current to be applied to the motor. The electrically conductive components of the circuit board of the above tool may be shielded from the supersaturated steam of the autoclave environment. When this tool is sterilized, these components are not adversely affected.

One or more of the printed circuit boards may also include one or more sensors that monitor the state of the tool motor, such as one or more hall sensors. To facilitate the responsiveness of the sensors, portions of the shell may be formed from material through which the physical quantity (quantities) monitored by the sensors can pass. For example, if one or more of the sensors monitors a magnetic field (fields) adjacent sections of the shell may be formed from combinations of magnetic and non-magnetic material that focuses the field (fields). If the sensors monitor photonic energy (light) the shell may have panels or sections of panels that are transparent to the wavelength of the monitored light.

Another configuration of the surgical power tool assembly 798 is illustrated in FIG. 24. The surgical power tool assembly 798 includes a variety of surgical attachments 822a, 822b, 822c, 822d that may be coupled to the housing 800. The surgical attachments 822a, 822b, 822c, 822d may each include an input shaft to receive torque from the motor of the surgical power tool assembly 798. Actuation of a trigger 810 may cause the motor to be powered by the battery 808 to drive a coupled surgical attachments 822a, 822b, 822c, 822d. The surgical attachments 822a, 822b, 822c, 822d may be used for pin driving, saw cutting, drilling, and other surgical operations.

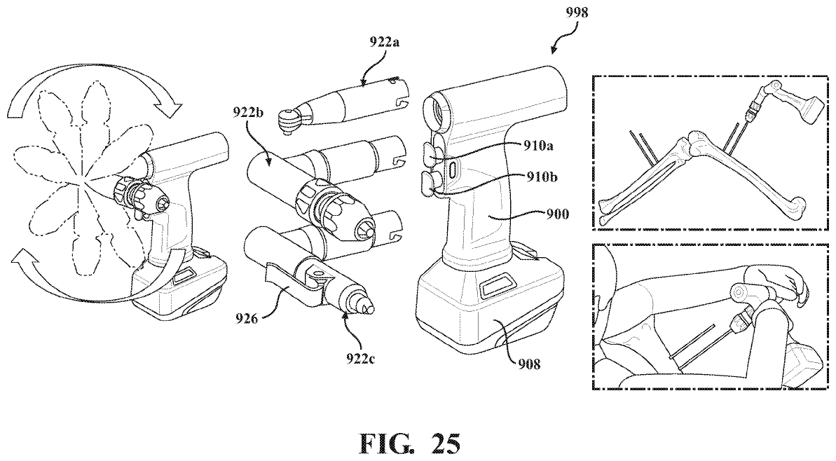

Another configuration of the surgical power tool assembly 898 is illustrated in FIG. 25. The surgical power tool assembly 898 includes a variety of surgical attachments 922a, 922b, 922c that may be coupled to the housing 900. The surgical attachments 922a, 922b, 922c may each include an input shaft to receive torque from the motor of the surgical power tool assembly 898. Actuation of one of the triggers 910a, 910b may cause the motor to be powered by the battery 908 to drive a coupled surgical attachments 922a, 922b, 922c. The surgical attachments 922a, 922b, 922c, 922d may be used for pin driving, saw cutting, drilling, and other surgical operations. The surgical attachments 922a, 922b, 922c may be configured such that the input shaft of the surgical attachment 922a, 922b, 922c is offset from the tool coupler by at least 45 degrees, at least 60 degrees, or by approximately 90 degrees. The feature can be further understood where the input shaft defines an input axis and the tool coupler also defines an axis, and the input axis is offset from the tool coupler axis by at least 45 degrees, at least 60 degrees, or by approximately 90 degrees. The lever 926 may be offset from the input axis so that the lever may be more conveniently accessed during placements of the pin.

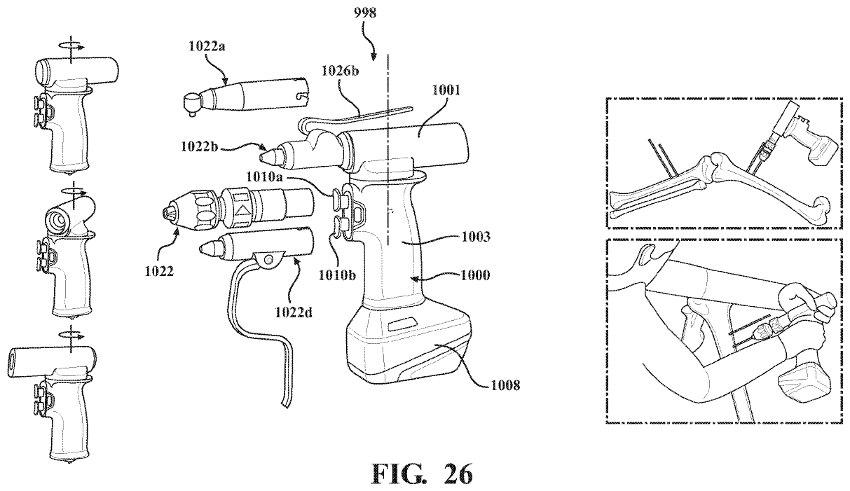

Another configuration of the surgical power tool assembly 998 is illustrated in FIG. 26. The surgical power tool assembly 998 includes a variety of surgical attachments 1022a, 1022b, 1022c, 1022d that may be coupled to the housing 1000. The surgical attachments 1022a, 1022b, 1022c, 1022d may each include an input shaft to receive torque from the motor of the surgical power tool assembly 998. Actuation of one of the triggers 1010a, 1010b may cause the motor to be powered by the battery 1008 to drive a coupled surgical attachment 1022a, 1022b, 1022c, 1022d. The surgical attachments 1022a, 1022b, 1022c, 1022d may be used for pin driving, saw cutting, drilling, and other surgical operations. The housing 1000 includes a handle portion 1003 and a barrel portion 1001. The handle portion 1003 extends along an axis and the barrel portion is configured to articulated about the handle portion to achieve a plurality of possible orientations of the surgical attachment 1022a, 1022b, 1022c, 1022d relative to the housing 1000. In other words, in this configuration, the barrel portion 1001 may articulate at various angles relative to the handle portion 1003. The housing portions 1001, 1003 may include suitable detent mechanisms, such as spring-loaded pins engaging preset holes, to fix the position of the barrel portion 1001 relative to the handle portion 1003.

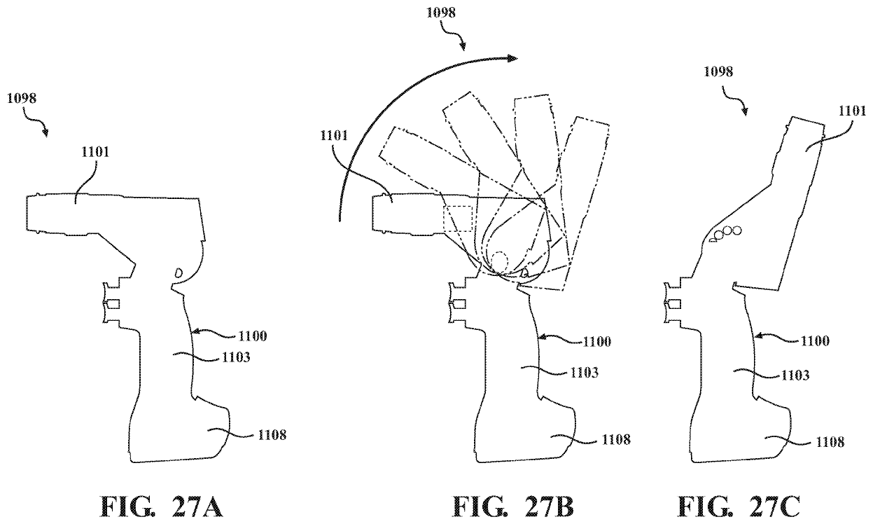

Another configuration of the surgical power tool assembly 1098 is illustrated in FIG. 27. The surgical power tool assembly 1098 may include a variety of surgical attachments that may be coupled to the housing 1000. The surgical attachments may each include an input shaft to receive torque from the motor of the surgical power tool assembly 1098. Actuation of one of the triggers may cause the motor to be powered by the battery 1108 to drive a coupled surgical attachment. The surgical attachments may be used for pin driving, saw cutting, drilling, and other surgical operations. The housing 1100 includes a handle portion 1103 and a barrel portion 1101. The handle portion 1103 extends along an axis and the barrel portion 1101 is configured to pivot relative the handle portion 1103 to achieve a plurality of possible orientations of the surgical attachment relative to the housing 1100. In other words, in this configuration, the barrel portion 1101 may pitch at various angles relative to the handle portion 1001. The housing portions 1101, 1103 may include suitable detent mechanisms, such as spring-loaded pins engaging preset holes, to fix the position of the barrel portion 1101 relative to the handle portion 1103.

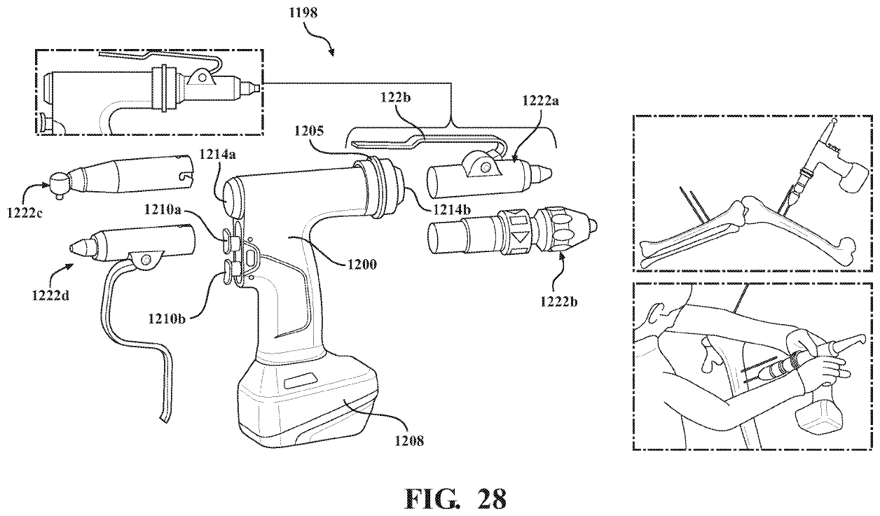

Another configuration of the surgical power tool assembly 1198 is illustrated in FIG. 28. The surgical power tool assembly 1198 includes a variety of surgical attachments 1222a, 1222b, 1222c, 1222d that may be coupled to the housing 1200. The surgical attachments 1222a, 1222b, 1222c, 1222d may each include an input shaft to receive torque from the motor of the surgical power tool assembly 1198. Actuation of one of the triggers 1210a, 1210b may cause the motor to be powered by the battery 1208 to drive a coupled surgical attachment 1222a, 1222b, 1222c, 1222d. The surgical attachments 1222a, 1222b, 1222c, 1222d may be used for pin driving, saw cutting, drilling, and other surgical operations. The surgical power tool assembly 1198 may include a mode switch 1205 movably coupled to the housing 1200 and movable between a first position and a second position. As implemented, the mode switch 1205 includes a ring-shaped member that generally surrounds the barrel of the housing 1200 and which is movable about the barrel portion of the housing 1200 to switch modes. The barrel portion may include a hall effector sensor to determine a position of the mode switch 1205. Alternatively, the mode switch 1205 may be located on other locations of the surgical power tool assembly 1198, or on a separate device, such as a tablet connected thereto.

The controller is operable to control the motor in a first mode when the mode switch 1205 is in a first position and operable to control the motor in a second mode when the mode switch 1205 is in a second position. The barrel portion of the housing 1200 may comprise a proximal region and a distal region. The housing 1200 includes a first attachment coupler 1214b in the proximal region and a second attachment coupler 1214a in the distal region.

The surgical power tool assembly 1198 may be designed to engage (e.g., be coupled to) two of the surgical attachments 1222a, 1222b, 1222c, 1222d simultaneously. A first surgical attachments 1222a, 1222b, 1222c, 1222d may be configured to be driven from the motor when the first attachment is engaged with the first attachment coupler 1214b. A second surgical attachment 1222a, 1222b, 1222c, 1222d may be configured to be driven from the motor when the second attachment is engaged with the second attachment coupler 1214a. The motor, when in the first mode, is operable to drive the first attachment and when in the second mode, the motor is operable to drive the second attachment. The motor may include a transmission selectively coupled to two or more separate output shafts. The first motor mode may drive the first output shaft and the second motor mode may drive the second output shaft. The first output shaft may be configured to drive the first attachment and the second output shaft may be configured to drive the second attachment. Alternatively, the tool may include two motors, with the mode switch 1205 controlling which motor is engaged depending on the position of the mode switch 1205.

The first attachment coupler 1214b is on an opposite side of the housing 1200 from the second attachment coupler 1214a. The second attachment may be operable to drive a surgical pin and the first attachment may be operable to drive a surgical saw blade or a drill bit or a screwdriver.

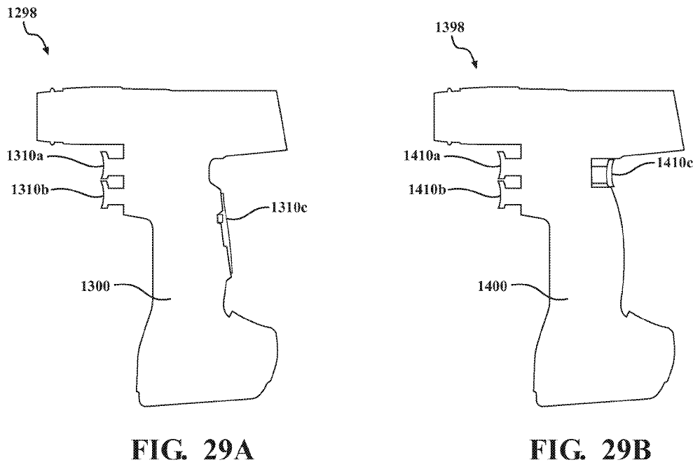

Alternative surgical power tool assemblies 1298, 1398 are shown in FIGS. 29A and 29B. The tool assemblies 1298, 1398 may include a housing 1300, 1400, an electric motor positioned in the housing 1300, 1400, a coupler operatively actuated by the electric motor, and a controller, optionally positioned within the housing 1300, 1400. The tool assemblies may also include a removable battery that is configured to supply power to the controller and/or the electric motor. The power tool may further include first and second triggers 1310a, 1310b, 1410a, 1410b movably coupled to the housing 1300, 1400 such that the controller is configured to turn on the electric motor when either of the first and second triggers 1310a, 1310b, 1410a, 1410b are depressed. The surgical power tool assemblies 1298, 1398 may further include a third trigger 1310c, 1410c that is movably coupled to the housing 1300, 1400 such that the controller is configured to turn on the electric motor when the third trigger 1310c, 1410c is depressed and/or contacted. In some implementations, actuation of any of the triggers causes the electric motor to rotate in a first direction, i.e., the same direction. In other implementations, one of the triggers may cause the electric motor to rotate in another direction than one of the other triggers. The first and second triggers 1310a, 1310b, 1410a, 1410b are positioned on the power tool assemblies 1298, 1398 such that they are accessible from a different grip than the third trigger 1310c, 1410c.

The housing 1300, 1400 may include a barrel portion and a handle portion, and the first and second triggers 1310a, 1310b, 1410a, 1410b may be disposed on a first region of the handle portion and the third trigger 1310c, 1410c may be disposed on a second region of the handle portion. The first region may be located opposite the second region relative to the handle portion, such that the triggers are on opposite sides of the handle portion. In some instances, the first region may be closer to the barrel portion than the second region.

Several configurations have been discussed in the foregoing description. However, the configurations discussed herein are not intended to be exhaustive or limit the invention to any particular form. The terminology which has been used is intended to be in the nature of words of description rather than of limitation. Certain inventive aspects of the present disclosure are made with reference to the following exemplary clauses:
I. A pin driver attachment system, the pin driver attachment comprising: an attachment housing to be removably coupled to a handpiece housing including a motor; an input shaft defining features for receiving torque from the handpiece and a first bore and a second bore, the first bore being coaxial with the second bore, the first bore being smaller than the second bore; a coupler coupled to the input shaft, the coupler movable between an engaged position and an unengaged position, wherein in the engaged position, a drill bit can be secured in the coupler, and in the unengaged position, the drill bit can be released from the coupler.
II. The pin drive attachment system of clause I, wherein first bore includes a plurality of flats, and the second bore includes a plurality of flats, and further comprising a surgical pin including at least six flats.
III. The pin driver attachment system of clause II, further comprising a surgical pin including a ferrous material.
IV. A powered surgical instrument, comprising: a housing defining an inner cavity therein, the housing including a handle portion; a body portion extending distally from the handle portion; a tool assembly disposed at a distal end of the body portion; a control circuit and a drive motor disposed within the inner cavity, wherein the drive motor is mechanically coupled to the tool assembly and the control circuit is configured to control the operation of the drive motor, wherein the control circuit includes a plurality of field effect transistors; and a potting material disposed in the inner cavity encapsulating at least a portion of the field effect transistors of the control circuit.
V. The powered surgical instrument of clause IV, further comprising a metal heat sink contacting at least two of the plurality of field effect transistors.
VI. The powered surgical instrument of clause IV, wherein the potting material is a polymer selected from the group consisting of polyesters, silicones, rubbers, epoxies, nylons, polyphthalamides, liquid crystal polymers, and combinations thereof.
VII. The powered surgical instrument according to clause VI, wherein the potting material includes a thermally conductive additive.
VIII. A powered surgical instrument, comprising: a housing defining an inner cavity therein, the housing including a handle portion; a body portion extending distally from the handle portion; a tool assembly disposed at a distal end of the body portion; a control circuit and a drive motor disposed within the inner cavity, wherein the drive motor is mechanically coupled to the tool assembly and the control circuit is configured to control the operation of the drive motor, wherein the control circuit includes a plurality of field effect transistors; and a metal heat sink contacting at least two of the plurality of field effect transistors.
IX. The powered surgical instrument of clause VIII, further comprising a potting material disposed in the inner cavity encapsulating at least a portion of the field effect transistors of the control circuit.
X. A method for manufacturing a powered surgical device, the method comprising: providing an enclosure of the powered surgical device, the enclosure defining a cavity, and a control circuit disposed in the enclosure, injecting a liquid potting material into the cavity of the sealed enclosure to hermetically seal the enclosure, the control circuit including: a circuit board; a motor controller coupled to the circuit board, and optionally, one or more field effect transistors coupled to the circuit board, and optionally one more trigger sensors and one or more motor sensors, such as one or more hall sensors, and solidifying the potting material to encapsulate at least a portion of the control circuit, such as encapsulating one more field effect transistors, the motor controller, motor sensors, trigger sensors and/or the circuit board.
XI. A surgical power tool assembly, comprising: a housing; an electric motor positioned in the housing; a coupler operatively actuated by the electric motor; a controller positioned in the housing; a battery configured to supply electrical power to the controller, the battery coupled to the housing, a trigger movably coupled to the housing such that the controller is configured to turn on the electric motor when the trigger is depressed and adjust a speed of the electric motor based on how far the trigger is depressed; wherein the coupler defines an axis; and wherein the trigger defines an actuation surface and moves between an actuated position and an unactuated position, and wherein the actuation surface is closer to the axis defined by the coupler in the actuated position than the unactuated position.
XII. The surgical power tool assembly of clause XI, further comprising a surgical attachment, the surgical attachment including an input shaft and a tool coupler, wherein the input shaft is offset from the tool coupler by at least 45 degrees, at least 60 degrees, or by approximately 90 degrees.
XIII. The surgical power tool assembly of clause XII, wherein the input shaft defines an input axis, and wherein the input axis is offset from the tool coupler by at least 45 degrees, at least 60 degrees, or by approximately 90 degrees.
XIV. The surgical power tool assembly of clause XIII, wherein the tool coupler is operable to engage a surgical pin, wherein the tool coupler includes a lever operable to move the tool coupler between a secured configuration and an unsecured configuration, wherein the lever is offset from the input axis.
XV. A surgical power tool assembly, comprising: a housing including a barrel portion and a handle portion; an electric motor positioned in the housing; a coupler operatively actuated by the electric motor; a controller positioned in the housing, a battery configured to supply electrical power to the controller, the battery coupled to the housing; a trigger movably coupled to the handle portion of the housing such that the controller is configured to turn on the electric motor when the trigger is depressed; wherein the handle portion defines a longitudinal axis, and wherein the barrel portion of the housing is operable to rotate about the longitudinal axis of the handle portion.
XVI. The surgical power tool assembly of clause XV, wherein the barrel portion defines an attachment coupler configured to receive a surgical attachment.
XVII. The surgical power tool assembly of clause XV, wherein the barrel portion includes a coupler to engage a surgical end effector.
XVIII. A surgical power tool assembly, comprising: a housing including a barrel portion and a handle portion; an electric motor positioned in the housing, a coupler operatively actuated by the electric motor; a controller positioned in the housing; a battery configured to supply electrical power to the controller, the battery coupled to the housing; a trigger movably coupled to the handle portion of the housing such that the controller is configured to turn on the electric motor when the trigger is depressed; wherein the handle portion defines an axis, and wherein the barrel portion of the housing is operable to rotate about an axis of the handle portion.
XIX. The surgical power tool assembly of clause XVIII, wherein the axis is perpendicular to a longitudinal axis of the handle portion.
XX. A surgical power tool assembly, comprising: a housing comprising a proximal region and a distal region, the housing including a first attachment coupler in the proximal region and a second attachment coupler in the distal region; an electric motor positioned in the housing; a controller positioned in the housing; a battery configured to supply electrical power to the controller, the battery coupled to the housing; a trigger movably coupled to the housing and the controller is configured to turn on the electric motor when the trigger is depressed; a mode switch movably coupled to the housing and movable between a first position and a second position, and the controller is operable to control the motor in a first mode when the mode switch is in a first position and operable to control the motor in a second mode when the mode switch is in a second position; a first attachment configured to be driven from the motor when the first attachment is engaged with the first attachment coupler; a second attachment configured to be driven from the motor when the second attachment is engaged with the second attachment coupler wherein the motor is in the first mode, the motor is operable to drive the first attachment and wherein the motor is in the second mode, the motor is operable to drive the second attachment.
XXI. The surgical power tool assembly of clause XX, wherein the first attachment coupler is on an opposite side of the housing from the first attachment coupler.
XXII. The surgical power tool assembly of clause XX, wherein the second attachment is operable to drive a surgical pin, and wherein the first attachment is operable to drive a surgical saw blade or drill bit.
XXIII. The surgical power tool assembly of clause XX, wherein the mode switch is a ring switch that is operable to rotate relative to the housing to switch between the first position and the second position.
XXIV. A surgical power tool assembly, comprising: a housing; an electric motor positioned in the housing; a coupler operatively actuated by the electric motor; a controller positioned in the housing; a battery configured to supply electrical power to the controller, the battery coupled to the housing; a first trigger movably coupled to the housing such that the controller is configured to turn on the electric motor when the first trigger is depressed; a second trigger movably coupled to the housing such that the controller is configured to turn on the electric motor when the second trigger is depressed, wherein the first trigger is accessible from a different grip than the second trigger.
XXV. The surgical power tool assembly of clause XXIV, wherein the housing includes a barrel portion and a handle portion, with the first trigger is on a first region of the handle portion and the second trigger is on a second region of the handle portion.
XXVI. The surgical power tool assembly of clause XXV, wherein the first region is located opposite the second region.
XXVII. The surgical power tool assembly of clause XXV, wherein the first region is close to the barrel portion than the second region.
XXVIII. The surgical power tool assembly of clause XXIV, further comprising a mode switch movably coupled to the housing and movable between a first position and a second position, and the controller is operable to control the motor in a first mode when the mode switch is in a first position and operable to control the motor in a second mode when the mode switch is in a second position; the housing comprising a proximal region and a distal region, the housing includes a first attachment coupler in the proximal region and a second attachment coupler in the distal region, a first attachment configured to be driven from the motor when the first attachment is engaged with the first attachment coupler; a second attachment configured to be driven from the motor when the second attachment is engaged with the second attachment coupler, wherein the motor is in the first mode, the motor is operable to drive the first attachment and wherein the motor is in the second mode, the motor is operable to drive the second attachment.
XXIX. The surgical power tool assembly of clause XXVIII, wherein the first trigger is operable to control the speed of the motor when the motor is in the first mode and the second trigger is operable to control the speed of the motor when the motor is in the second mode.
XXX. A surgical power tool assembly, comprising: a housing; an electric motor positioned in the housing; a coupler operatively actuated by the electric motor; a controller positioned in the housing; a battery configured to supply electrical power to the controller, the battery coupled to the housing; a trigger movably coupled to the housing such that the controller is configured to turn on the electric motor when the trigger is depressed and adjust a speed of the electric motor based on how far the trigger is depressed; wherein the coupler defines an axis; and wherein the trigger defines an actuation surface, the trigger moveable between an actuated position and an unactuated position; and wherein the actuation surface is closer to the axis defined by the coupler in the actuated position than the unactuated position.
XXXI. The surgical power tool assembly of clause XXX, wherein the housing defines a trigger axis, and wherein the trigger has a pivoting end and a free end, the trigger being pivotable about the trigger axis at its free end.
XXXII. The surgical power tool assembly of clause XXX, wherein the housing includes a base and a protrusion extending from the base, with the protrusion extending the actuation surface when the trigger is in the actuated position and the unactuated position.
XXXIII. The surgical power tool assembly of clause XXX, wherein the trigger includes a run-safe switch.
XXXIV. The surgical power tool assembly of clause XXX, wherein the trigger includes a first and second magnets.
XXXV. A powered surgical tool comprising: a handpiece defining a void space; an electric motor disposed in the void space; a first printed circuit board disposed in the void space and being rigid, the first circuit board including trigger sensors and motor control sensors; a second printed circuit board disposed in the void space and being rigid; a third circuit board disposed in the void space and being rigid; each of the first circuit board, the second circuit board, and third circuit board defining a plane, with the planes of the first, second and third circuit boards being parallel to one another; a controller configured to regulate power drawn from a power source based on user input, the controller being mounted to one of the second printed circuit board and the third circuit board.
XXXVI. A surgical power tool assembly for driving a surgical pin, comprising: a surgical handpiece comprising: a handpiece housing, an electric motor positioned in the handpiece housing, a controller positioned in the handpiece housing, a trigger moveably coupled to the handpiece housing, the trigger having an actuation surface moveable between an actuated position and an unactuated position, wherein the controller is configured to control a speed of the motor based on the actuated position of the trigger, and an output drive coupled to the electric motor and configured to be rotated about an axis by the electric motor, wherein the actuation surface is closer to the axis in the actuated position than in the unactuated position; and a pin driver attachment for driving the surgical pin.
XXXVII. A method of driving pins in a robotically-assisted knee arthroplasty procedure, the method including: providing a battery-powered surgical tool, coupling a surgical attachment to the battery-powered surgical tool, drilling a hole in at least two locations with a drill bit using a first drive portion of the surgical attachment, driving a first pin in one of the at least two locations with a second drive portion of the surgical attachment, and driving a second pin having a different diameter than the first pin in another of the at least two locations with a third drive portion of the surgical attachment.

1-46. (canceled)

47. A method of inserting a pin with a surgical power tool, the surgical power tool including a trigger and a lever coupled to a collet, the method comprising:
grasping a housing of the surgical power tool with a user's hand;
engaging the lever with an index finger of the user's hand to clamp a surgical pin with the collet;
engaging the trigger with a finger different from the index finger of the user's hand;
driving the surgical pin into bone with the surgical power tool; and
grasping the housing of the surgical power tool with a hammer grip.

48. (canceled)

49. The method of claim 48, wherein the step of grasping is further defined as grasping the housing such that a user's thumb is closer to a proximal end of the surgical power tool than a distal end of the surgical power tool.

50. The method of claim 47, wherein the step of engaging the trigger further comprises depressing the trigger to adjust a speed of an electric motor based on a position of the trigger.

51. The method of claim 50, further comprising the step of engaging the lever with the index finger while a user's thumb is closer to a battery of the surgical power tool assembly than the index fingers.

52. The method of claim 47, wherein the steps of depressing the lever and depressing the trigger are performed simultaneously.

53. The method of claim 51 or wherein the collet includes a jaw, and wherein the step of depressing the lever further comprises depressing the lever to move the jaw from an unclamped state to a clamped state in which the jaw engages the surgical pin.

54. The method of claim 47, wherein the step of grasping the housing further comprises grasping the housing such that a battery is disposed above the user's hand during use.

55-58. (canceled)

59. A method of inserting a surgical pin with a surgical power tool assembly, wherein the surgical power tool assembly includes a housing, an electric motor positioned in the housing, a coupler operatively actuated by the electric motor, a controller positioned in the housing, a battery removably coupled to the housing and configured to supply electrical power to the controller, a trigger movably coupled to the housing, and a surgical attachment to engage the surgical pin including a lever separate from the trigger, the method including:
inserting the surgical pin into the coupler;
grasping the housing with a user's hand such that a user's thumb is closer to the battery than the coupler;
depressing the trigger with a user's finger while the user's thumb is closer to the battery than the coupler to adjust a speed of the electric motor;
depressing the lever with the user's finger while the user's thumb is closer to the battery than the coupler;
driving the surgical pin into a bone with the surgical power tool assembly.

60. The method of claim 59, further comprising drilling a hole into the bone with a twist drill coupled to the coupler.

61. The method of claim 59, wherein the steps of depressing the lever and depressing the trigger are performed with different fingers.

62. The method of claim 61, wherein the steps of depressing the lever and depressing the trigger are performed simultaneously.

63. The method of claim 59, wherein the surgical attachment comprises a collet with a jaw, and wherein the step of depressing the lever further comprises depressing the lever to move the jaw from an unclamped state to a clamped state in which the jaw engages the surgical pin.

64. A method of inserting a surgical pin with a surgical power tool assembly, wherein the surgical power tool assembly includes a housing, an electric motor positioned in the housing, a coupler operatively actuated by the electric motor, a controller positioned in the housing, a battery removably coupled to the housing and configured to supply electrical power to the controller, a trigger movably coupled to the housing, the method including:
inserting the surgical pin into the coupler;
grasping the housing with a user's hand such that a user's thumb is closer to the battery than the coupler and such that the battery is disposed above the user's hand during use;
engaging the trigger with a user's finger while the user's thumb is closer to the battery than the coupler; and
driving the surgical pin into a bone with the surgical power tool assembly.

65. The method of claim 64, further comprising drilling a hole into the bone with a twist drill coupled to the coupler.

66. The method of claim 64, wherein the step of engaging the trigger further comprises depressing the trigger to adjust a speed of the electric motor.

67. The method of claim 66, wherein the surgical power tool assembly further includes a surgical attachment to engage the surgical pin, wherein the surgical attachment includes a lever separate from the trigger and, the method further comprising the step of depressing the lever with the user's finger while the user's thumb is closer to the battery than the coupler.

68. The method of claim 67, wherein the steps of depressing the lever and depressing the trigger are performed with different fingers.

69. The method of claim 68, wherein the steps of depressing the lever and depressing the trigger are performed simultaneously.

70. The method of claim 67, wherein the surgical attachment comprises a collet with a jaw, and wherein the step of depressing the lever further comprises depressing the lever to move the jaw from an unclamped state to a clamped state in which the jaw engages the surgical pin.
